(19) United States
(12) Patent Application Publication
(10) Pub. No.: US 2009/0222071 A1
(43) Pub. Date: Sep. 3, 2009

(54) Self-insulated thermal packs for thermal therapy

(76) Inventors: YaQi Li, New Hope, PA (US); Lijiang Mao, Beijing (CN)

Correspondence Address: YaQi Li, 526 Clydesdale Drive, New Hope, PA, 18938, US

(21) Appl. No.: 12/072,949

(22) Filed: Feb. 29, 2008

(51) Int. Cl. A61F 7/02

(52) U.S. Cl. 607/114

(57) ABSTRACT

A therapeutic thermal pack with built-in seal and insulation functions for preserving the thermal condition of its thermal module. The thermal pack comprises a plurality of thermal module for thermal therapy, an insulation cover comprising a fastening means for attaching the thermal module to a body part, and a closing means for enclosing the thermal module inside the insulation cover for preserving the temperature of the thermal module. When it is configured for storage and carry, this thermal pack can maintain its therapeutic condition for an extended time period without using an additional insulation. It can thus be conveniently carried to the sports fields using any regular bag. When the pack is used for a treatment, the built-in insulation functions minimize the heat exchange between the thermal modules of the thermal pack and the surrounding environment and thus prolong the treatment time of the thermal pack.

### FIELD OF THE INVENTION

This invention generally relates to thermal packs used for therapeutic applications. More specifically, embodiments of the invention construct a new class of thermal pack with built-in seal and insulation functions for preserving the therapeutic condition of the thermal pack.

### BACKGROUND

Therapeutic applications for thermal packs are well-known. Cold therapy is commonly used for reducing bruising, bleeding, and inflammation of the human body caused by traumatic injuries or excessive physical activity. Heat therapy is frequently used to relieve certain pain or soreness of the human body, such as joint pain associated with arthritis.

Many prior art thermal packs have been developed for thermal therapy. A typical thermal pack includes at least one thermal component that is primarily responsible for the heat exchange between the thermal pack and the body part being treated. Such a thermal component is referred to as the thermal module of the thermal pack in this application.

A thermal module can be anything that has substantial thermal capacity and can be heated up or cooled down to a desired temperature. However, it should preferably have the flexibility to conform to the surface contour of the body part being treated.

Gel packs are commonly used in thermal packs as the thermal modules. Gel packs are typically plastic bags filled with gel-like liquid and are still pliable when frozen. Examples of such gel packs are introduced in U.S. Pat. Nos. 3,885,403 and 5,129,391.

Packages of granular materials, such as natural whole grains or beans, may also be used as the thermal module of a thermal pack. By including aromatic materials in the granular materials, such thermal modules may be conveniently incorporated with functions for aromatherapy.

For example, U.S. Pat. No. 6,699,271 introduces a therapeutic wrap containing a plurality of narrow channels filled with Basmati rice and aromatic herbs. The rice and herbs, after being cooled or heated to a desired temperature, are primarily responsible for the heat exchange between the wrap and the treated body when the wrap is in use. In this case, the channels filled with the rice and herbs are the thermal modules of the wrap with incorporated functions for aromatherapy. More examples of adding aromatic functions to the thermal modules can be seen in U.S. Pat. Nos. 5,476,492, 5,603,727 and 5,948,010.

A thermal module may have different shapes. It may be in the form of a package, such as a gel pack or a bean bag, or the form of a sheet, such as the sheet-form pack disclosed by U.S. Pat. No. 6,544,284.

A thermal module may comprise apparatus for generating thermal reactions and thus can achieve a desired temperature by itself. For example, heating pads that employ a one-time chemical reaction such as catalyzed rusting of iron have been used in various applications. A reusable sodium acetate heat pad is also known. Such an acetate heat pad contains a super-saturated solution of sodium acetate. A small flat disc of notched ferrous metal is embedded in the solution. Pressing the disc releases very tiny adhered crystals of sodium acetate into the solution and triggers the crystallization of the solution. The heat released from the solution during the crystallization is thus used for heat therapy.

For another example, U.S. Pat. No. 6,393,843 introduces a thermal pack comprising a solvent and two solutes that are chemically separated in different compartments until time of use. The thermal reaction produced by the combination of the solvent and solutes can be either endothermic or exothermic, so the thermal pack can be used to apply cold or heat therapy, respectively.

A thermal pack may comprise apparatus for attaching and holding the thermal modules to various body parts. To increase the comfort and prevent cold/heat bums to the treated body, heat-resistant sheet materials may be used between the thermal module and the body. To reduce water condensation on the surface of the thermal module, water-absorbing and waterproof sheet materials may also be used to cover the thermal module. These aspects have been addressed by many prior art thermal packs, such as those introduced by U.S. Pat. Nos. 5,716,388 and 5,415,624.

The prior art thermal packs are not intended to preserve their therapeutic condition by themselves. Without a built-in insulation function particularly for preserving the temperature of their thermal module, the prior art thermal packs need to be stored or carried using an external thermally insulated container, typically a bulky cooler. It is thus inconvenient to have these packs available for thermal therapy when the users are away from home.

The thermal packs disclosed in this application have built-in seal and insulation functions particularly for preserving the thermal conditions of their thermal modules. Therefore, these thermal packs can maintain their therapeutic condition for an extended time period, such as several hours, without using any additional insulation. Such self-insulation functions provide two major advantages for the applications of the thermal packs:

First, the insulation functions enhance the therapeutic efficiency of the thermal packs. Without sufficient thermal insulation, the thermal module may have a considerable heat exchange with the surrounding environment when a thermal pack is applied to the body being treated. This heat exchange does not contribute to any therapeutic effects for the body, but reduces the therapeutic life of the thermal module. The built-in insulation functions effectively minimize this unwanted heat exchange and thus prolong the therapeutic life of the thermal module.

Secondly, the built-in self-insulation functions make it more conveniently for general users to carry a thermal pack to where they need. This is particularly beneficial for the application of cold therapy. It is well known that cold therapy is most effective when it is applied immediately after injury or activity. The longer the delay before treatment, the more swelling, bruising and inflammation, and the longer recovery time. Therefore, it is always desirable to have the cold treatment immediately available during and after sports participation. With built-in self-insulation functions, the thermal packs of this invention can maintain their therapeutic condition for an extended time period without requiring an additional container and thus be carried in any regular sports bag, such as a tennis racquet bag, to the sports fields.

In accordance with the invention, three embodiments are disclosed in this application as examples to illustrate the principle components of the new thermal packs. These principle components include a plurality of thermal modules for providing thermal therapy, an insulation cover comprising a fastening means for attaching the thermal module to a body part, and a closing means for enclosing the thermal module inside the insulation cover for preserving the temperature of the thermal module.

The thermal packs of this invention can be either used for cold therapy or heat therapy depending on the therapeutic requirement. For improving the therapeutic effect of the thermal packs, other functions may be added to the thermal pack, including functions for aromatherapy and/or magnetic treatment. For the convenience and safety for the users, other components may also be added to the thermal pack, such as a temperature indicator for showing the temperature of the thermal modules and a timing device for monitoring the treatment time.

### SUMMARY

This invention discloses a class of new thermal pack that has both seal and insulation functions built in it for preserving the thermal condition of its thermal module. The thermal pack comprises a plurality of thermal module for thermal therapy, an insulation cover comprising a fastening means for attaching the thermal module to a body part, and a closing means for enclosing the thermal module inside the insulation cover for preserving the temperature of the thermal module. When the thermal pack is in use for thermal therapy, the insulation cover protects a portion of the thermal module for minimizing the unwanted heat exchanges between the thermal module and the surrounding environment. The therapeutic life of the thermal pack is thus prolonged. When the pack is configured for storage or carrying, the thermal module is enclosed inside a closed and thermally insulated environment provided by the insulation cover. The therapeutic condition of the thermal modules can thus be preserved for an extended time period without requiring additional or external insulation.

The above and other embodiments are mentioned not to limit or define the invention, but to provide examples of embodiments of the invention to aid understanding thereof. Embodiments are discussed in the Detailed Description, and further description of the invention is provided there. Advantages offered by the various embodiments of the invention may be further understood by examining this specification.

### BRIEF DESCRIPTION OF THE FIGURES

These and other features, aspects, and advantages of the present invention are better understood when the following Detailed Description is read with reference to the accompanying drawings, wherein:

FIGS. 1A-1B depict perspective views of a thermal pack used for elbow treatment, according to an embodiment of the invention;

FIGS. 2A-2B depict cross profiles of the thermal pack used for elbow treatment, according to an embodiment of the invention;

FIG. 3A depicts a portion of the thermal pack used for elbow treatment, according to an embodiment of the invention;

FIGS. 4A-4B depict a thermal pack used for general body parts, according to an embodiment of the invention;

FIG. 5 depicts an example of applying a thermal pack on a body part;

FIGS. 6A-6B depict a thermal pack used for general body parts, according to an embodiment of the invention;

### DETAILED DESCRIPTION

Embodiments and accompanying drawings depicted in the following are provided by way of example to illustrate some general principles of the invention, and should not be construed as limiting the scope of the invention in any manner. One skilled in the art would also recognize that various changes and modifications could be made herein, without departing from the principles and scope of the invention.

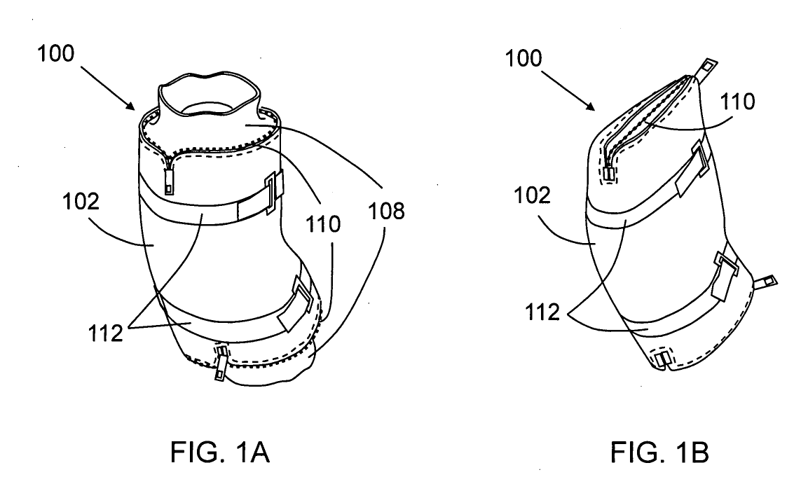

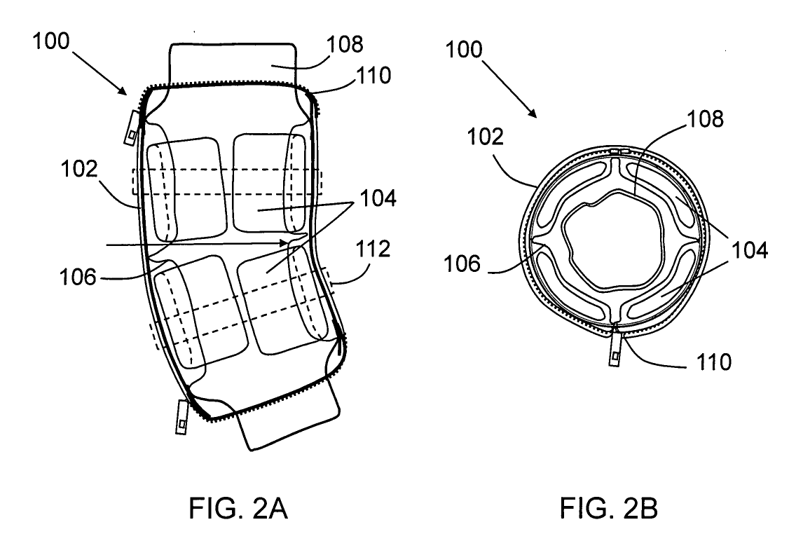

According to an embodiment of the invention, a thermal pack 100 used as a thermal sleeve for elbow treatment (termed “thermal sleeve” herein) is demonstrated in FIGS.1-3. FIGS. 1A-1B and 2A-2B illustrate the external views and the cross sections of the thermal sleeve 100. As shown in FIGS. 1A-1B, the thermal sleeve 100 comprises, a thermal insulation cover 102 (termed “insulation cover” herein); two rib cuffs 108 (termed “cuffs” herein); two zippers 110; and two fastening belts 112. As shown in FIGS. 2A-2B, the thermal sleeve 100 further comprises a plurality of thermal module 104 and a lining 106.

As shown in FIGS. 1-2, the insulation cover 102 constructs a tubular sleeve body. The lining 106 is installed under the insulation cover 102. The thermal module 104, which may be a gel pack according to an embodiment of the invention, is installed between the insulation cover 102 and the lining 106 (installation method will be described later). The cuffs 108 are connected to the lining 106 and can be pushed into and pulled out of the sleeve body on both ends. The zippers 110 are installed at the edges of the insulation cover 102 on both ends. The fastening belts 112 are installed on the external surface of the insulation cover 102.

As the zippers 110 are unzipped with the cuffs 108 being pulled out from both ends of the sleeve body, as shown in FIG. 1A, the thermal sleeve 100 can be readily put on an arm at the elbow position. In such a circumstance, the insulation cover 102 and the cuffs 108 minimize the heat transfer between the thermal module 104 and the surrounding outside environment, enhancing the energy efficiency of the thermal module for elbow treatment. By changing the tension of the fastening belt 112, the contact pressure between the thermal module 104 and the arm, and thus the intensity of the thermal therapy, may be adjusted.

When the zippers 110 are zipped with the cuffs 108 being pushed inside the sleeve body on both ends, as shown in FIG. 1B, the insulation cover 102 constructs a closed and thermally insulated environment for the thermal module 104 inside the cover. Therefore, the heat transfer between the thermal module 104 and the outside environment is minimized. In this configuration for carrying or storage, the temperature of the thermal module 104, and thus the therapeutic condition of the thermal sleeve 100, can be preserved for an extended time period.

According to the embodiment shown in FIGS. 2A-2B, eight thermal modules 104 may be installed surrounding the arm on both the upper and lower sides of the elbow. According to other embodiments of the invention, a plurality of the thermal module 104 may be installed only in particular positions inside the insulation cover 102. For example, the thermal module 104 may be installed only for one side of the arm.

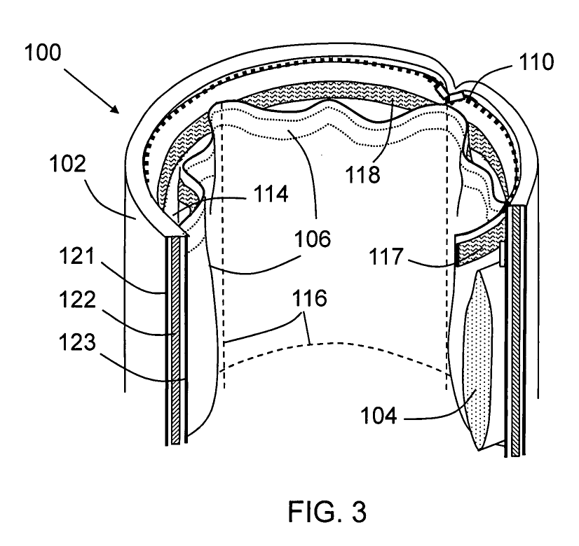

FIG. 3 illustrates a method of installing the thermal module 104 for the thermal sleeve 100, according to an embodiment of the invention. The figure depicts the upper half section of the thermal sleeve 100 without showing the cuffs 108.

As shown in FIG. 3, a plurality of pocket 114 may be constructed by connecting the lining 106 and the insulation cover 102 along the stitch line 116. The pocket 114 may be designed to have an open side, thus a thermal module 104 can be inserted or removed from the pocket conveniently. The opening of the pocket 114 may be sealed using Velcro straps 117 and 118 installed on the insulation cover 102 and the lining 106 respectively as shown in the figure.

The method illustrated in FIG. 3 for installing the thermal module 104 may have various changes or modifications according to other embodiments of the invention. For example, the lining 106 may not be employed and the thermal module 104 may be enclosed in a separate bag made by sheet materials. The bag may be attached to the inside of the insulation cover 102 using Velcro straps or the snap buttons, the thermal module 104 can thus be installed on or removed from the thermal sleeve 100 conveniently.

The insulation cover 102 may comprise a plurality of layers. According to an embodiment of the invention, as shown in FIG. 3, the insulation cover 102 comprises a surface layer 121, an insulation layer 122 and a support layer 123. The surface layer 121 may preferably be made of durable fabrics, such as Neoprene fabric. The support layer 123 may also be made from durable fabrics, such as Nylon fabrics. The insulation layer 122 may be made of material with low thermal conductivity, such as flexible thermoplastic foams. Both the lining 106 and the cuffs 108 may preferably be made by Nylon fabric or knitting.

To freeze or heat the thermal module in the thermal sleeve 100, users may remove the thermal module 104 from the sleeve, or, alternatively, simply place the sleeve into a freezer or a microwave oven. The zippers 110 may be unzipped and the cuffs 108 may be kept open to reduce the freezing time of the thermal module if the thermal sleeve is put in a freezer. As the thermal module reach a desired temperature and are sealed inside the insulation cover 102 (with the zippers 110 on both ends being closed), the thermal sleeve 100 is ready to be carried to the sports fields in any sports bag.

The thermal sleeve 100 is introduced above as an example for illustrating the general principles and the functions of the principle module of the invention. It is within the scope of this invention that various changes and modifications can be made suitable for therapeutic applications on other body parts.

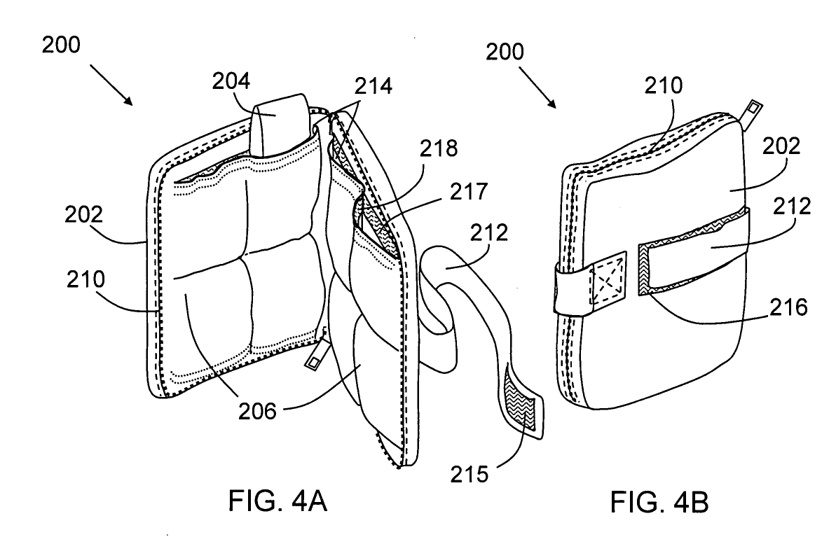

As an example, a thermal pack constructed as a folder (termed “thermal folder” herein) is demonstrated in FIGS. 4A-4B according to an embodiment of the invention. The thermal folder 200 comprises an insulation cover 202; a plurality of thermal module 204; a lining 206, a zipper 210; and a fastening belt 212.

As shown in FIG. 4A, the insulation cover 202 of the thermal folder 200 constructs a flat pad, which can be folded in the middle. The cover is flexible and can be bent to conform to the surface contour of the body. A plurality of pocket 214 is formed by the insulation cover 202 and the lining 206 inside the folder. The pocket 214 may have one side open, which may be closed using the Velcro straps 217 and 218 installed on the edge of the pocket. The thermal module 204 can thus be put and sealed inside the pocket 214.

The fastening belt 212, which is preferably made of stretchable material, such as woven braided elastic materials, is installed on the insulation cover 202 of the thermal folder 200. For connecting the fastening belt 212 to the insulation cover 202, Velcro straps 215 and 216 may be installed on the belt and the cover respectively.

The zipper 210 is installed at the edge of the insulation cover 202. When the insulation cover 202 is closed as shown in FIG. 4B, the thermal module 204 are thermally insulated inside the cover. Under this configuration, the temperature of the thermal module 204 may be preserved for an extended time period while the thermal folder 200 is carried or stored without any additional insulation.

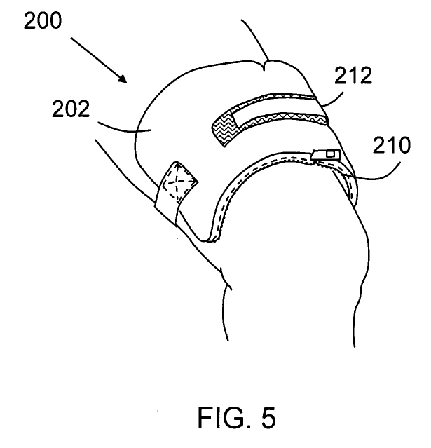

When the zipper 210 is unzipped as shown in FIG. 4A, the thermal folder 200 can be opened into a flat pad and applied to a body part for thermal therapy. An example of using the thermal folder 200 for treating the thigh of a human body is illustrated in FIG. 5. The fastening belt 212 may hold the pad in position as shown in the figure.

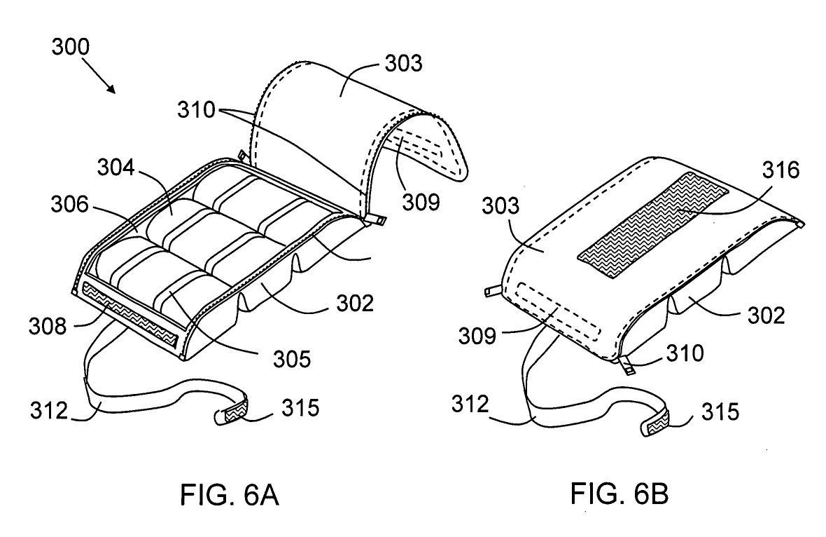

FIGS. 6A-6B illustrate a thermal pack 300 (termed “thermal pad” herein) for treating a general body part, according to yet another embodiment of the invention. The thermal pad 300 comprises a plurality of thermal module 304, an insulation cover 302 which constructs a container 306 and a lid 303 for enclosing the thermal module.

The thermal pad 300 also comprises elastic straps 305 for holding the thermal module 304 in the container 306. The thermal module 304 is preferably a gel pack covered with a sheet material, such as nylon, and may be removed from the container 306 conveniently. Also, the thermal module 304 and the container 306 are preferably flexible so they can be bent to conform to the body part being treated.

The thermal pad 300 also comprises two zippers 310 and a fastening belt 312. The zippers 310 are installed at the edges of the container 306 and the lid 303, so that the lid 303 may be closed to cover (as shown in FIG. 6B) or opened to expose (as shown in FIG. 6A) the thermal module 304. Velcro straps 308 and 309 are installed on the container 306 and the lid 303 respectively for providing a good seal for the thermal module 304 when the lid 303 is closed.

The fastening belt 312, Velcro straps 315 and 316, may hold the thermal pad 300 to the body part being treated. With the lid 303 open, the thermal pad 300 can be used to treat a body part in the similar fashion shown in FIG. 5.

While the present invention has been described in terms of preferred and alternative embodiments, it will be obvious to one skilled in the art that many alternations and modifications may be made without substantially departing from the spirit of the invention. Following are some examples of such alternations and modifications.

Instead of using the zipper, the insulation cover of the thermal pack may be closed using other closing means, such as those using snap buttons, Velcro straps or tie strings.

Instead of using gel packs, the thermal module of the thermal pack may use a package of granular materials or a module that can achieve a desired temperature by means of thermal reactions. The thermal module may also be added with aromatic and/or magnetic materials, so that the thermal pack may have supplementary functions for aromatherapy and/or magnet therapy.

For monitoring the therapeutic conditions of the thermal pack, a temperature indicator may be added to the thermal pack for displaying the temperature inside the insulation cover. For monitoring the treatment time, a timing device may also be attached to the thermal pack.

An additional insulation means, such as an external insulation bag or container, may be used with the thermal pack for preserving the therapeutic conditions of the thermal packs for a longer time period.

The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed is:

1. A therapeutic thermal pack comprising:
a plurality of thermal module;
a cover comprising a plurality of sheet material and a fastening means for placing said thermal module in close proximity to a body part;
a closing means for enclosing said thermal module inside said cover for preserving the temperature of said thermal module.

2. The apparatus of claim 1 wherein the thermal module is a gel pack.

3. The apparatus of claim 1 wherein the thermal module comprises granular material.

4. The apparatus of claim 1 wherein the thermal module comprises aromatic material.

5. The apparatus of claim 1 wherein the thermal module comprises magnetic material.

6. The apparatus of claim 1 wherein the cover comprises a installation means for placing the thermal module inside the cover

7. The apparatus of claim 1 wherein the cover constructs a tubular structure.

8. The apparatus of claim 1 wherein the cover constructs a foldable flat pad.

9. The apparatus of claim 1 wherein the cover constructs a container with a covering lid.

10. The apparatus of claim 1 wherein the closing means comprises using a zipper.

11. The apparatus of claim 1 wherein the closing means comprises using a Velcro strap.

12. The apparatus of claim 1 wherein the closing means comprises using a tie string.

13. The apparatus of claim 1 wherein the closing means comprises using a button.

14. The apparatus of claim 1 wherein the closing means comprises wrapping the thermal module using the cover.

15. The apparatus of claim 1 wherein the thermal pack comprises a component for showing the temperature of the thermal module.

16. The apparatus of claim 1 wherein the thermal pack comprises a component for counting the treatment time.

17. A method for constructing a therapeutic thermal pack comprising:
a plurality of thermal module;
a cover comprising a plurality of sheet material and a fastening means for placing said thermal module in close proximity to a body part;
a closing means for enclosing said thermal module inside said cover for preserving the temperature of said thermal module.

18. The method of claim 17 wherein the cover constructs a tubular structure.

19. The method of claim 17 wherein the cover constructs a foldable flat pad.

20. The method of claim 17 wherein the cover constructs a container with a covering lid.
